SEQUENCE LISTING (23 sequences)

SEQ ID NO: 1 - protein, 430 aa, Lycopersicon esculentum
MGSEKMMKINIKESTLVKPSKPTPTKRIWSSNLDLIVGRIHLLTVYFYKPNGSSNFFDNKVIKEALSNVLVSFYPMAGRLGRDEQGRIEVNCNGEGVLFVEAESDSCVDDFGDFTPSLELRKLIPSVETSGDISTFPLVIFQITRFKCGGVALGGGVFHTLSDGLSSIHFINTWSDIARGLSVAVPPFIDRTLLRARDPPTYSFEHVEYHPPPTLNSSKNRESSTTTMLKFSSEQLGLLKSKSKNEGSTYEILAAHIWRCTCKARGLPEDQLTKLHVATDGRSRLCPPLPPGYLGNVVFTATPIAKSCELQSEPLTNSVKRIHNELIKMDDNYLRSALDYLELQPDLSTLIRGPAYFASPNLNINSWTRLPVHECDFGWGRPIHMGPACILYEGTIYIIPSPNSKDRNLRLAVCLDAGHMSLFEKYLYEL

SEQ ID NO: 2 - protein, 436 aa, Nicotiana tabacum
MGSEKMMKINIKESTLVKPSKPTPTKRLWSSNLDLIVGRIHLLTVYFYKPNGSSNFFDSKIMKEALSNVLVSFYPMAGRLARDEQGRIEINCNGEGVLFVEAESDAFVDDFGDFTPSLELRKLIPTVDTSGDISTFPLIIFQVTRFKCGGVSLGGGVFHTLSDGLSSIHFINTWSDIARGLSVAIPPFIDRTLLRARDPPTSSFEHVEYHPPPSLISSSKSLESTSPKPSTTTMLKFSSDQLGLLKSKSKHDGSTYEILAAHIWRCTCKARALSDDQLTKLHVATDGRSRLCPPLPPGYLGNVVFTGTPMAKSSELLQEPLTNSAKRIHSALSKMDDNYLRSALDYLELLPDLSALIRGPTYFASPNLNINSWTRLPVHDSDFGWGRPIHMGPACILYEGTVYILPSPNSKDRNLRLAVCLDADHMPLFEKYLYEF

SEQ ID NO: 3 - DNA, 1456 bp, Nicotiana tabacum
atgggaagtgaaaaaatgatgaaaattaatatcaaggaatcaacattagtaaaaccatcaaaaccaacaccaacaaaaagactttggagttctaacttagatttaatagtgggaagaattcatcttttaacagtatatttctataaaccaaatggatcttcaaatttctttgattcaaaaataatgaaagaagcattaagtaatgttcttgtttcattttacccaatggctggaagattagctagagatgaacaaggaagaattgagataaattgtaatggagaaggagttttatttgttgaagctgaaagtgatgcttttgttgatgattttggtgattttactccaagtttggaacttaggaaacttattcctactgttgacacttctggtgatatttctactttccccctcatcatctttcaggttactcgtttcaaatgtggtggagtttcacttggtggaggagtattccacactttatcagatggtctctcatcaattcacttcatcaacacatggtccgatatagcccgaggcctctccgtcgccatcccgccgttcatcgaccggaccctcctccgtgcacgggacccaccaacatcgtctttcgagcacgtcgagtatcatcctcctccatctctaatttcatcatcaaaaagcttagaatccactagcccaaagcctagtaccacaaccatgttaaaattctctagtgaccaacttgggcttctaaagtccaagtccaaacatgatggtagcacttacgaaatcctcgcggcccatatttggcgttgcacgtgcaaggcacgtgcactgtccgacgatcaattgaccaaattacatgtggccactgatggtaggtctaggctttgccctcctttgccaccaggttacttaggaaatgttgtgttcacaggcacacctatggcaaaatcaagtgaacttttacaagaaccattgacaaattcagccaagagaattcatagtgcattatcaaaaatggatgacaattacctaagatcagctctcgattacctcgaattactgcccgatttatcggctttaatccgtggaccgacgtactttgctagccctaatcttaatattaatagttggactagattgcctgttcatgattcagattttggatggggaaggccaattcatatgggaccagcttgcattttatatgaagggacagtttatatattgccaagtccaaatagtaaagataggaacttgcgtttggctgtttgtttagatgctgatcacatgccactatttgagaagtatttgtatgaattttgagaggttgaaaaaaaaatcaagaatgttccaacacttgagaattatcttaggtgtgggtggttttggattaaggcattttgtaacttgttttctattgtttttttggggggtcagtttgttttcaaaaaaaaaaaaaaaaaaaaaa

SEQ ID NO: 4 - DNA, 1293 bp, Lycopersicon esculentum
atgggaagtgaaaaaatgatgaaaattaatatcaaagaatcaacactagtgaaaccatcaaaaccaacaccaacaaagagaatttggagttctaatttggatttaattgttggaagaattcatcttttgactgtttatttttataaaccaaatggatcttcaaatttttttgataataaagttattaaagaagcattaagtaatgttttagtttcattttatccaatggctggaagattaggtagggatgaacaaggtagaattgaagttaattgtaatggtgaaggtgttttgtttgttgaggctgaaagtgattcatgtgttgatgattttggtgattttacaccatctttggaacttagaaaactcattccaagtgttgaaacctctggagatatctcaactttcccactagttatatttcagattactcgtttcaagtgtggcggagtcgctcttggtggtggagtattccacacgttatccgatggtctctcatccatccacttcatcaacacgtggtcggacatcgcccgtggcctctccgtcgcagtcccgccgttcatcgatcggacgctcctccgtgcaagggacccaccgacatattctttcgagcacgttgagtaccatcctccacctaccctaaactcatcgaaaaatcgcgagtccagtaccacgaccatgttgaaattctcgagtgaacaactcgggcttcttaagtccaagtccaaaaatgagggtagcacctatgaaatcctcgcagcccatatttggcgatgcacgtgcaaggcacgtggattgccagaggatcaattgaccaaattacacgtggccaccgacggaaggtcaaggctttgccctcccttgccaccgggttacctaggaaacgtcgtgttcacggcaaccccaatagctaaatcatgcgaacttcaatcagagccgttgacaaattccgtcaagagaattcacaacgagttgatcaaaatggacgacaattacctaagatcagcactggattacctcgaattacaacctgatttatcaaccctaattcggggcccggcttactttgctagccctaacctcaatattaatagttggactaggttgcctgtccatgagtgtgattttggatggggtaggccaattcatatgggaccagcttgcattttatatgaagggacaatttatattataccaagtccaaattctaaagataggaacttgcgtttggctgtttgtctagatgctggtcacatgtcactatttgaaaaatatttatatgaattatga

SEQ ID NO: 5 - DNA, 35 bp, Artificial sequence
  Oligonucleotide
gactcgagtcgacatcgattttttttttttttttt

SEQ ID NO: 6 - DNA, 35 bp, Artificial sequence
  Oligonucleotide
ccatgggaagtgaaaaaatgatgaaaattaatatc

SEQ ID NO: 7 - DNA, 35 bp, Artificial sequence
  Oligonucleotide
ggatcctcataattcatataaatatttttcaaata

SEQ ID NO: 8 - DNA, 29 bp, Artificial sequence
  Oligonucleotide
gagcacgtcgagtatcatcctcctccatc

SEQ ID NO: 9 - DNA, 32 bp, Artificial sequence
  Oligonucleotide
ctaatttcatcatcaaaaagcttagaatccac

SEQ ID NO: 10 - DNA, 17 bp, Artificial sequence
  Oligonucleotide
gactcgagtcgacatcg

SEQ ID NO: 11 - DNA, 32 bp, Artificial sequence
  Oligonucleotide
gtggattctaagctttttgatgatgaaattag

SEQ ID NO: 12 - DNA, 53 bp, Artificial sequence
  Oligonucleotide
ggggacaagtttgtacaaaaaagcaggctcagattactcgtttcaagtgtggc

SEQ ID NO: 13 - DNA, 54 bp, Artificial sequence
  Oligonucleotide
ggggaccactttgtacaagaaagctgggttgataaatcaggttgtaattcgagg

SEQ ID NO: 14 - DNA, 51 bp, Artificial sequence
  Oligonucleotide
ggggacaagtttgtacaaaaaagcaggctcaaatgtggtggagtttcactg

SEQ ID NO: 15 - DNA, 51 bp, Artificial sequence
  Oligonucleotide
ggggaccactttgtacaagaaagctgggtcgggcagtaattcgaggtaatc

SEQ ID NO: 16 - protein, 5 aa, Artificial sequence
  Motif
DFGWG

SEQ ID NO: 17 - protein, 4 aa, Artificial sequence
  Consensus sequence of the CoA-transferase family
SNLD

SEQ ID NO: 18 - protein, 4 aa, Artificial sequence
  Consensus sequence of the CoA-transferase family
AGRL

SEQ ID NO: 19 - protein, 4 aa, Artificial sequence
  Consensus sequence of the CoA-transferase family
FVEA

SEQ ID NO: 20 - protein, 5 aa, Artificial sequence
  Consensus sequence of the CoA-transferase family
FKCGG

SEQ ID NO: 21 - protein, 4 aa, Artificial sequence
  Consensus sequence of the CoA-transferase family
RSRL

SEQ ID NO: 22 - protein, 4 aa, Artificial sequence
  Consensus sequence of the CoA-transferase family
PPLP

SEQ ID NO: 23 - protein, 4 aa, Artificial sequence
  Consensus sequence of the CoA-transferase family
YLRS

(19) United States
(12) Patent Application Publication
(10) Pub. No.: US 2006/0150268 A1
(43) Pub. Date: Jul. 6, 2006

(54) Plant-derived transferase genes

(76) Inventors: Catherine Rosemary Martin, Norwich (GB); Anthony Michael, Norwich (GB); Ricarda Niggeweg, Heidelberg (DE)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/518,884

(22) PCT Filed: Jun. 17, 2003
(86) PCT No.: PCT/GB03/02645

§ 371 Date: Sep. 8, 2005

(30) Foreign Application Priority Data
Jun. 21, 2002 (GB) 0214406.1

(51) Int. Cl. A01H 1/00; C07H 21/04; C12N 9/10; C12N 15/82; C12N 5/04

(52) U.S. Cl. 800/278; 435/468; 435/419; 435/193; 536/23.2

(57) ABSTRACT

The invention discloses methods for controlling chlorogenic acid synthesis by manipulation of hydroxycinnamoyl-CoA quinate hydroxycinnamoyl transferase (HQT) genes. Isolated nucleic acids encoding HQT and methods for their use are provided. Preferred embodiments are the nucleotide sequences which encode the polypeptide sequences of FIG. 3 (sequences of FIG. 7). Also provided are variant sequences (e.g. alleles and orthologues) and complementary sequences, plus vectors, host cells and plants. Methods of the invention include the use of nucleic acids to express or down-regulate HQT in plant cells and plants. The methods may be used to alter one or more characteristics in a plant e.g. texture, flavour and antioxidant properties.

### TECHNICAL FIELD

The present invention relates to methods and materials for controlling chlorogenic acid synthesis based on manipulation of hydroxycinnamoyl-CoA quinate hydroxycinnamoyl transferase (HQT) genes.

### BACKGROUND ART

The plant phenylpropanoid pathway is responsible for the synthesis of a wide variety of secondary metabolic compounds, including lignins, salicylates, coumarins, hydroxycinnamic amides, flavonoid phytoalexins, pigments, UV light protectants and antioxidants (DIXON and PAIVA, 1995). Phenylpropanoids have a range of important functions in plants, such as structural components (lignin), protectants against biotic and abiotic stresses (antipathogenic phytoalexins, antioxidants and UV-absorbing compounds), pigments (anthocyanins) and signalling molecules (flavonoid nodulation factors; STRACK, 1997). In addition to their role in structure and protection of the plants, the compounds of the phenylpropanoid pathway also have an important effect on plant qualities, such as texture, flavour, colour and processing characteristics (MACHEIX et al., 1991).

Chlorogenic acid, an ester of quinic and caffeic acid (STOECKIGT and ZENK, 1974), is a major compound of the phenylpropanoid pathway in Coffea and Solanaceous species such as tobacco, tomato and sweet potato. The levels of chlorogenic acid are constitutively high in some species (Coffea), while in others the biosynthesis of chlorogenic acid can be induced by a variety of stimuli, such as high light (KUEHNL et al., 1987, GRACE et al., 1998), UV radiation (DEL MORAL, 1972), wounding (RHODES and WOOLTORTON, 1978), and fungal elicitors (YAO et al., 1995).

The fact that chlorogenic acid is one of the initial products formed during the transcriptional activation of the phenylpropanoid pathway by pathogen infection and abiotic stress, led to the assumption that chlorogenic acid acts as a carbon reservoir. The mobilization of this pool to form downstream phenylpropanoid products such as lignins, phytoalexins and cell wall cross-linking agents would enable the plants to respond immediately to pathogen attack (YAO et al., 1995, DIAZ et al., 1997).

Recent studies proposed another potential function of chlorogenic acid in plants—as an antioxidant. Environmental stresses such as high light, UV light, low temperature and pathogen infection can all lead to an increase of reactive oxygen species in plants. To avoid lethal damage caused by these oxidative species, plants have developed a complex antioxidant system. In addition to the well-characterised system which includes enzymes such as superoxide dismutase or ascorbate peroxidase, a few studies have analysed the role of phenolic metabolites in scavenging reactive oxygen species (GRACE and LOGAN, 2000). Chlorogenic acid has been shown to have antioxidant activity (CASTELLUCCIO et al., 1995, KONO et al., 1997). Polyphenols such as chlorogenic acid are ideal for free-radical scavenging activities due to the hydrogen-donating activity of their catechol groups and have been shown to be more effective antioxidants in vitro than vitamine E and C (RICE-EVANS et al., 1997).

Free radicals and other reactive species are considered to be important factors in the development of cancer and cardiovascular diseases. Due to their antioxidant capacity the interest in food polyphenolics such as chlorogenic acid has increased. Food phenolics are thought to provide beneficial implications in human health, e.g. in the treatment and prevention of cancer (BRAVO, 1998, RICE-EVANS et al., 1997).

Two different pathways have been proposed for the biosynthesis of chlorogenic acid. In sweet potato the enzyme hydroxycinnamoyl-D-glucose quinate hydroxycinnamoyl transferase has been isolated (VILLEGAS and KOJIMA, 1986). This catalyses the transfer of the caffoyl moiety of caffeoyl-D-glucose to quinic acid.

An alternative pathway has been proposed on the basis of chlorogenic acid biosynthesis in tobacco cell suspension cultures initially described by STOECKIGT and ZENK (1974). This involves transesterification between caffeoyl-CoA thiolesters and quinic acid. The enzyme catalysing this reaction, hydroxycinnamoyl-CoA quinate hydroxycinnamoyl transferase (HQT) has not yet been isolated.

Recently, the isolation of a 3-hydroxylase from Arabidopsis, which has been shown to catalyze the formation of chlorogenic acid by hydroxylation of coumaroyl quinic acid in vitro, led to the consideration that plants may also be able to synthesize chlorogenic acid by hydroxylation (SCHOCH et al., 2001).

Although HQT has been partially purified from various plant species, particularly in Solanaceae (RHODES and WOOLTORTON, 1976, RHODES et al., 1979, ULBRICH and ZENK, 1979, LOFTY et al., 1992, LOFTY et al., 1994), the gene has not been isolated.

Indeed, to date 116 transferases have been identified in plants (examples include anthranilate N-hydroxycinnamoyl/benzoyl transferase, that catalyses the first committed reaction of phytoalexin biosynthesis, deacetylvindoline 4-O-acetyltransferase and trichothecene 3-O-acetyltransferase). Nevertheless, apart from the DFGWG sequence motif, there are only few conserved amino acids within the transferase family. Even within the subgroup of anthranilate N-hydroxycinnamoyl/benzoyl transferase the amino acid sequences differ so much that the enzymes from Arabidopsis and clove pink are not closely related (FIG. 4). This suggests that it is very difficult to correlate the function of a transferase with the sequence information provided. Hence, cloning a transferase of known function is far from straightforward.

In seeking to solve the problem of providing the HQT gene, the inventors originally thought the tobacco hypersensitive response protein 201 would be a good candidate for HQT because of its expression pattern during hypersensitive response after pathogen attack. Hsr201 is a member of the transferase family and shows less homology to known anthranilate transferase, which indicates a putative new function in tobacco. However, the recombinant protein expressed in E. coli showed no HQT enzyme activity (FIG. 5).

The isolation and characterization of the gene and enzyme HQT that catalyses the synthesis of chlorogenic acid, the major antioxidant in higher plants, may contribute inter alia to improvements in the antioxidant content of food. It could further be used to modify inter alia, this and other traits in plants.

### DISCLOSURE OF THE INVENTION

The inventors have succeeded in cloning HQT genes from plants. This is the first time a gene known to encode an HQT has been cloned.

In order to provide the HQT genes, briefly, the inventors purified HQT from tobacco leaves by using anion exchange and hydrophobic interaction chromatography (FIG. 1). As noted above, no isolated HQT had previously been published. The purification procedure gave a pure protein with HQT enzyme activity. The reaction product of the enzyme assay was identified by HPLC analysis as chlorogenic acid, the product of the esterification of quinic acid with caffeoyl-CoA.

Two partial amino acid sequences of the purified protein, obtained from Q-TOF analysis were found to match with a hydroxycinnamoyl-benzoyl transferase from sweet potato and the translation products of tobacco and tomato EST clones. By using overlapping tomato EST clones a full-length cDNA was assembled (see FIG. 2).

Using the sequence of the EST clones the full-length cDNA clones of HQT from tobacco and tomato were isolated by using RT-PCR. The two related cDNAs encode 430 amino acids (tomHQT, calculated mass 47,960 D) and 436 amino acids (tobHQT, calculated mass of 48,390 D). The amino acid sequences show 90% homology to each other and 81-83% homology to the N-hydroxycinnamoyl/benzoyl transferase from sweet potato (accession number AB035183). Although the activity and substrate specificity of the sweet potato protein has apparently not been characterised, the classification of the protein as an N-hydroxycinnamoyl/benzoyl transferase has been made according to sequence homologies to other members of the transferase family. In the light of the disclosure herein, it appears this enzyme may also have HQT activity.

The encoded protein sequences of both cDNA clones contain the DFGWG sequence motif that is characteristic of transferases from plants and may therefore form part of the active site.

To confirm the identity of proteins as HQT the enzyme activity was analysed after expressing the cDNA from tomato in E. coli. As shown in FIG. 5 the recombinant expressed protein showed HQT enzyme activity in contrast to the expression of hsr201 where no enzyme activity was detected.

Increased HQT enzyme activity was also detected in leaves of Nicotiana benthamiana plants expressing high levels of tomato and tobacco HQT under the control of the 35S promoter (FIG. 6). This increase in enzyme activity leads to a 2-fold higher amount of chlorogenic acid in vivo in plants indicating that the synthesis of chlorogenic acid might not be as strictly regulated as previously assumed and that the substrates are not limiting.

In various aspects of the invention the HQT gene, or its variants, may be employed in the regulation of chlorogenic acid synthesis in a plant. Regulation of chlorogenic acid synthesis may be used inter alia to alter the flavour, palatability, texture, nutritional value or antioxidant properties of the plant.

According to an aspect of the present invention there is provided an isolated nucleic acid molecule encoding a polypeptide which has HQT activity (i.e. an HQT).

As described above, HQT catalyses the production of chlorogenic acid via transfer of the caffeoyl moiety of caffeoyl-CoA to quinic acid. Such activity can be assessed, for example, using the assay described in Example 2. Briefly, the assay uses caffeoyl CoA and quinic acid as substrates, and the rate of conversion of caffeoyl CoA to chlorogenic acid is determined by spectrophotometry.

Nucleic acid according to the present invention may include cDNA, RNA, genomic DNA and modified nucleic acids or nucleic acid analogs (e.g. peptide nucleic acid). Where a DNA sequence is specified, e.g. with reference to a figure, unless context requires otherwise the RNA equivalent, with U substituted for T where it occurs, is encompassed.

Likewise, DNA is generally found in double-stranded form, and the complementary strand of such DNA sequences is also included in the ambit of the invention. A nucleic acid is “the complement” of another nucleic acid to which it is complementary. ‘Complementary to’ means that the sequence is capable of base pairing with the coding sequence whereby A is the complement of T (and U); G is the complement of C; and may be of equal length to, or of a portion of, said DNA sequence.

Nucleic acid molecules according to the present invention may be provided in isolated and/or purified from their natural environment, in substantially pure or homogeneous form, or free or substantially free of other nucleic acids of the species of origin. Where used herein, the term ‘isolated’ encompasses all of these possibilities. The nucleic acid molecules may be wholly or partially synthetic. In particular they may be recombinant in that nucleic acid sequences which are not found together in nature (do not run contiguously) have been ligated or otherwise combined artificially. Alternatively they may have been synthesised directly e.g. using an automated synthesiser.

Most preferably the nucleic acid is derived from tobacco or tomato. Thus included in this aspect of the invention, there are also provided nucleic acids encoding the polypeptides of FIG. 3. Such nucleic acids may have the sequences of FIG. 7, or may consist or comprise the coding portion thereof (such as that underlined in FIG. 7b).

In this aspect of the present invention there are also provided nucleic acids which are variants of the said sequences. A variant nucleic acid molecule shares homology with, or is identical to, all or part of the coding sequence discussed above. Generally variants may encode, or be used to isolate or amplify nucleic acids which encode, polypeptides which have HQT activity as described above. However, variants which do not encode polypeptides having HQT activity are also provided, eg. for use in probing or silencing. Variants of the present invention can be artificial nucleic acids, which can be prepared by the skilled person in the light of the present disclosure. Alternatively they may be novel, naturally occurring, nucleic acids, isolatable using the sequences of the present invention.

Sequence variants which occur naturally may include HQT alleles (which typically include polymorphisms or mutations at one or more bases) or pseudoalleles (which may occur at closely linked loci to the HQT gene). Also included within the scope of the present invention are isogenes, or other genes homologous to the HRT sequences of the invention and belonging to the same family as the HQT gene. Although these may occur at different genomic loci to the HQT gene, they typically share conserved regions with it.

Artificial variants (derivatives) may be prepared by those skilled in the art, for instance by site directed or random mutagenesis, or by direct synthesis. Preferably the variant nucleic acid is generated either directly or indirectly (e.g. via one or more amplification or replication steps) from an original nucleic acid having all or part of the sequence shown in FIG. 7.

Thus a variant may be a distinctive part or fragment (however produced) corresponding to a portion of the sequence provided. The fragments may encode particular functional parts of the polypeptide. Alternatively, the fragments may have utility in probing for, or amplifying, the sequence provided or closely related ones. Suitable lengths of fragment, and conditions, for such processes are discussed in more detail below.

Also included are nucleic acids corresponding to those above, but which have been extended at the 3′ or 5′ terminus.

The term ‘variant’ nucleic acid as used herein encompasses all of these possibilities. When used in the context of the present invention with regard to polypeptides or proteins, ‘variant’ indicates the encoded expression product of the variant nucleic acid.

Some of the aspects of the present invention relating to variants will now be discussed in more detail.

Similarity or homology (the terms are used interchangeably) or identity may be as defined and determined by the TBLASTN program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wisconsin 53711). Preferably sequence comparisons are made using MultAlin (see Corpet, 1988, Nucleic Acids Research 16 10881-10890). Default parameters are preferably set as follows:
Gap value (penalty for the first residue in a gap): −12
Gap length weight (penalty for additional residues in a gap): −2

Alternatively, sequence comparisons may be made using FASTA and FASTP (see Pearson & Lipman, 1988. Methods in Enzymology 183: 63-98). Parameters may be set, using the default matrix, as follows:
Gapopen (penalty for the first residue in a gap): −12 for proteins/−16 for DNA
Gapext (penalty for additional residues in a gap): −2 for proteins/−4 for DNA
KTUP word length: 2 for proteins/6 for DNA.

Homology may be at the nucleotide sequence and/or encoded amino acid sequence level. Preferably, the nucleic acid and/or amino acid sequence shares at least about 90%, 95%, 96%, 97%, 98% or 99% homology or identity.

Homology may be over the full-length of the relevant sequence shown herein, or may be over a part of it, preferably over a contiguous sequence of about or greater than about 20, 25, 30, 33, 40, 50, 67, 133, 167, 200, 233, 267, 300, 333, 400 or more amino acids or codons, compared with FIG. 3 respectively.

Thus a variant polypeptide in accordance with the present invention may include within either of the sequences shown in FIG. 3, a single amino acid or 2, 3, 4, 5, 6, 7, 8, or 9 changes, about 10, 15, 20, 30, 40 or 50 changes, or greater than about 50, 60, 70, 80 or 90 changes. In addition to one or more changes within the amino acid sequence shown, a variant polypeptide may include additional amino acids at the C-terminus and/or N-terminus. Naturally, changes to the nucleic acid which make no difference to the encoded polypeptide (i.e. ‘degeneratively equivalent’) are included.

The activity of functional variant polypeptides may be assessed by transformation into a host capable of expressing the nucleic acid of the invention. Methodology for such transformation is described in more detail below.

In a further aspect of the invention there is disclosed a method of producing a derivative nucleic acid comprising the step of modifying any of the sequences disclosed above, particularly the coding sequence of FIG. 7.

Alternatively changes to a sequence may produce a derivative by way of one or more of addition, insertion, deletion or substitution of one or more nucleotides in the nucleic acid, leading to the addition, insertion, deletion or substitution of one or more amino acids in the encoded polypeptide.

Such changes may modify sites which are required for post translation modification such as cleavage sites in the encoded polypeptide; motifs in the encoded polypeptide for glycosylation, lipoylation etc. Leader or other targeting sequences (e.g. membrane or golgi locating sequences) may be added to the expressed protein to determine its location following expression.

Other desirable mutation may be random or site directed mutagenesis in order to alter the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This may be so even when the substitution is in a region which is critical in determining the conformation of the peptide. Also included are variants having non-conservative substitutions.

As is well known to those skilled in the art, substitutions to regions of a peptide which are not critical in determining its conformation may not greatly affect its activity because they may not greatly alter the peptide's three dimensional structure. In regions which are critical in determining the peptides conformation or activity such changes may confer advantageous properties on the polypeptide. Indeed, changes such as those described above may confer slightly advantageous properties on the peptide e.g. altered stability or specificity. It may be preferred to retain the putative active site motif DFGWG (amino acid residues 382-386 of the tobacco HQT nucleotide sequence of FIG. 3)

In a further aspect of the present invention there is provided a method of identifying and/or cloning a nucleic acid variant from a plant which method employs an HQT sequence as described herein.

For instance, nucleotide sequence information provided herein may be used in a data-base (e.g. of ESTs, or STSs) search to find homologous sequences, and expression products of which can be tested for activity as described below in Example 2.

Nucleotide sequence information provided herein may be used to design probes and primers for probing or amplification of HQT or variants thereof. An oligonucleotide for use in probing or PCR may be about 32 or fewer nucleotides in length (e.g. 16, 18, 21 or 24). Generally specific primers are upwards of 14 nucleotides in length. For optimum specificity, primers of 16-32 nucleotides in length may be preferred. Those skilled in the art are well versed in the design of primers for use processes such as PCR.

Preferred primers which can be used include any of those shown in the Examples below as being suitable for amplifying HQT sequences.

If required, probing can be done with entire restriction fragments of the cDNA sequences disclosed herein, or the full-length cDNAs themselves. Fragments may be used e.g. the sequence employed may be about 100 nucleotides or more, about 200 nucleotides or more, about 300 nucleotides or more, or about 400 nucleotides or more, in each case the sequence may be a contiguous sequence selected from those nucleic acids disclosed herein.

Naturally sequences may be based on either FIG. 7, or the complement thereof. Small variations may be introduced into the sequence to produce ‘consensus’ or ‘degenerate’ primers if required.

Such probes and primers form one aspect of the present invention.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined. DNA for probing may be prepared from RNA preparations from cells. Probing may optionally be done by means of so-called ‘nucleic acid chips’ (see Marshall & Hodgson (1998) Nature Biotechnology 16: 27-31, for a review).

In one aspect, there is provided a method of obtaining an HQT variant nucleic acid molecule which comprises:
(a) providing a preparation of nucleic acid from a cell, e.g. from plant cells,
(b) providing a nucleic acid molecule which is a nucleic acid of the invention or probe or primer therefor,
(c) contacting nucleic acid in said preparation with said probe or primer under conditions for hybridisation, and,
(d) identifying said nucleic acid variant if present by its hybridisation with said nucleic acid molecule.

Test nucleic acid may be provided from a cell as genomic DNA, cDNA or RNA, or a mixture of any of these, preferably as a library in a suitable vector. If genomic DNA is used the probe may be used to identify untranscribed regions of the gene (e.g. promoters etc).

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include amplification using PCR (see below), RNase cleavage and allele specific oligonucleotide probing. The identification of successful hybridisation is followed by isolation of the nucleic acid which has hybridised, which may involve one or more steps of PCR or amplification of a vector in a suitable host.

Preliminary experiments may be performed by hybridising under low stringency conditions. For probing, preferred conditions are those which are stringent enough for there to be a simple pattern with a small number of hybridisations identified as positive which can be investigated further.

For example, hybridizations may be performed, according to the method of Sambrook et al. (below) using a hybridization solution comprising: 5×SSC (wherein ‘SSC’=0.15 M sodium chloride; 0.15 M sodium citrate; pH 7), 5×Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes−1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989): Tₘ=81.5° C.+16.6Log [Na+]+0.41 (% G+C)−0.63 (% formamide)−600/#bp in duplex

As an illustration of the above formula, using [Na+]=[0.368] and 50-% formamide, with GC content of 42% and an average probe size of 200 bases, the Tₘ is 57° C. The Tₘ of a DNA duplex decreases by 1−1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

It is well known in the art to increase stringency of hybridisation gradually until only a few positive clones remain. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

An alternative, which may be particularly appropriate with plant nucleic acid preparations, is a solution of 5×SSPE (final 0.9 M NaCl, 0.05M sodium phosphate, 0.005M EDTA pH 7.7), 5×Denhardt's solution, 0.5% SDS, at 65° C. overnight, (for high stringency, highly similar sequences). Washes in 0.2×SSC/0.1% SDS at 65° C. for high stringency.

In a further embodiment, hybridisation of a nucleic acid molecule to a variant may be determined or identified indirectly, e.g. using a nucleic acid amplification reaction, particularly the polymerase chain reaction (PCR). PCR requires the use of two primers to specifically amplify target nucleic acid, so preferably two nucleic acid molecules with sequences characteristic of HQT are employed. Using RACE PCR, only one such primer may be needed (see “PCR protocols; A Guide to Methods and Applications”, Eds. Innis et al, Academic Press, New York, (1990)).

Thus a method involving use of PCR in obtaining nucleic acid according to the present invention may include:
(a) providing a preparation of nucleic acid, e.g. from a plant cell,
(b) providing a pair of nucleic acid molecule primers for PCR, at least one of said primers being a primer according to the present invention as discussed above,
(c) contacting nucleic acid in said preparation with said primers under conditions for performance of PCR,
(d) performing PCR and determining the presence or absence of an amplified PCR product.

The presence of an amplified PCR product may indicate identification of a variant.

In all cases above, if need be, clones or fragments identified in the search may be extended. For instance if it is suspected that they are incomplete, the original DNA source (e.g. a clone library, mRNA preparation etc.) can be revisited to isolate missing portions e.g. using sequences, probes or primers based on that portion which has already been obtained to identify other clones containing overlapping sequence. As used herein, unless the context demands otherwise, the term HQT is intended to cover any of the nucleic acids of the invention described above, including functional variants.

The methods described above may also be used to determine the presence of one of the nucleotide sequences of the present invention within the genetic context of an individual plant, optionally a transgenic plant such as may be produced as described in more detail below. This may be useful in plant breeding programmes e.g. to directly select plants containing alleles which are responsible for desirable traits in that plant species, either in parent plants or in progeny (e.g hybrids, F1, F2 etc.). Thus use of markers which can be designed by those skilled in the art on the basis of the nucleotide sequence information disclosed herein, for selection of a gene encoding a polypeptide with HQT activity, forms one part of the present invention.

As used hereinafter, all such HQT-related nucleotide sequences discussed or obtainable as described above will be referred to as “HQT nucleotide sequences” or “HQT nucleic acids” unless context demands otherwise.

In a further aspect of the present invention, HQT nucleotide sequences are in the form of a recombinant and preferably replicable vector.

‘Vector’ is defined to include, inter alia, any plasmid, cosmid, phage or Agrobacterium binary vector in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication).

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

Specifically included are shuttle vectors by which is meant a DNA vehicle capable, naturally or by design, of replication in two different host organisms, which may be selected from actinomycetes and related species, bacteria and eucaryotic (e.g. higher plant, mammalian, yeast or fungal cells).

A vector including nucleic acid according to the present invention need not include a promoter or other regulatory sequence, particularly if the vector is to be used to introduce the nucleic acid into cells for recombination into the genome.

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

By “promoter” is meant a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment, the promoter is an inducible promoter.

The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Thus this aspect of the invention provides a gene construct, preferably a replicable vector, comprising a promoter (optionally inducible) operably linked to a nucleotide sequence provided by the present invention, such as the HQT gene or a variant thereof.

Particularly of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148). Suitable vectors may include plant viral-derived vectors (see e.g. EP-A-194809).

Suitable promoters which operate in plants include the Cauliflower Mosaic Virus 35S (CaMV 35S). Other examples are disclosed at pg 120 of Lindsey & Jones (1989) ‘Plant Biotechnology in Agriculture’ Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

The present invention also provides methods comprising introduction of a construct of the invention into a plant cell or a microbial cell and/or induction of expression of a construct within such cell, by application of a suitable stimulus e.g. an effective exogenous inducer.

The invention further encompasses a host cell transformed with nucleic acid or a vector according to the present invention (e.g. comprising an HQT nucleotide sequence) especially a plant or a microbial cell. In the transgenic plant cell (i.e. transgenic for the nucleic acid in question) the transgene may be on an extra-genomic vector or incorporated, preferably stably, into the genome. There may be more than one heterologous nucleotide sequence per haploid genome.

The term “heterologous” is used broadly in this aspect to indicate that the gene/sequence of nucleotides in question (e.g. encoding HQT) have been introduced into said cells of the plant or an ancestor thereof, using genetic engineering, i.e. by human intervention. A heterologous gene may replace an endogenous equivalent gene, i.e. one which normally performs the same or a similar function, or the inserted sequence may be additional to the endogenous gene or other sequence. Nucleic acid heterologous to a plant cell may be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

Thus a further aspect of the present invention provides a method of transforming a host cell involving introduction of a construct as described above into a host cell and causing or allowing recombination between the vector and the host cell genome to introduce a nucleic acid according to the present invention into the genome. Preferably, the host cell will be a plant cell.

The host cell (e.g. plant cell) is preferably transformed by the construct, which is to say that the construct becomes established within the cell, altering one or more of the cell's characteristics. Nucleic acid can be introduced into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11.

Agrobacterium transformation is widely used by those skilled in the art to transform dicotyledonous species.

Recently, there has also been substantial progress towards the routine production of stable, fertile transgenic plants in almost all economically relevant monocot plants (see e.g. Hiei et al. (1994) The Plant Journal 6, 271-282)). Microprojectile bombardment, electroporation and direct DNA uptake are preferred where Agrobacterium alone is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233).

The particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention.

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The generation of fertile transgenic plants has been achieved in the cereals rice, maize, wheat, oat, and barley (reviewed in Shimamoto, K. (1994) Current Opinion in Biotechnology 5, 158-162.; Vasil, et al. (1992) Bio/Technology 10, 667-674; Vain et al., 1995, Biotechnology Advances 13 (4): 653-671; Vasil, 1996, Nature Biotechnology 14 page 702).

Plants which include a plant cell according to the invention are also provided.

In addition to the regenerated plant, the present invention embraces all of the following: a clone of such a plant, seed, selfed or hybrid progeny and descendants (e.g. F1 and F2 descendants).

A plant according to the present invention may be one which does not breed true in one or more properties. Plant varieties per se may be excluded, particularly registrable plant varieties according to Plant Breeders' Rights.

The invention also provides a plant propagule from such plants, that is any part which may be used in reproduction or propagation, sexual or asexual, including cuttings, seed and so on. It also provides any part of these plants e.g. edible leaves which includes the plant cells or heterologous DNA described above.

The invention further provides a method of influencing or affecting chlorogenic acid synthesis in a plant, the method including the step of causing or allowing expression of a heterologous HQT nucleic acid sequence as discussed above within the cells of the plant.

The step may be preceded by the earlier step of introduction of the nucleic acid into a cell of the plant or an ancestor thereof.

In a preferred embodiment of the invention, plants may be stably transformed with a vector containing a HQT nucleotide sequence to increase chlorogenic acid synthesis within the plant's cells. Alternatively, transient expression of HQT in a plant may be achieved by, for example, infiltration of plant tissues with Agrobacterium tumefaciens containing a vector comprising a HQT nucleotide sequence. Non-limiting demonstration of both transient and stable expression is given below. For example, the inventors have stably transformed tobacco and tomato plants with a binary vector containing the cDNA of HQT from tobacco and tomato under the control of the double 35S promoter (Example 8). They have also demonstrated transient expression of HQT and concomitant increase in chlorogenic acid production in leaves of Nicotiana benthamiana, as set forth in Example 9.

Increasing chlorogenic acid production in stable transformed plants, preferably tobacco and tomato, may improve their antioxidant content, which is of significance for plants and also for nutrition. Plants overexpressing HQT may be produced and analysed under different environmental conditions, in order to address the antioxidant function of HQT in tomato and tobacco. Other traits which may be influenced include pathogen resistance and abiotic stress.

The foregoing discussion has been generally concerned with uses of the nucleic acids of the present invention for production of functional polypeptides, thereby increasing the amount of chlorogenic acid produced and hence the level of chlorogenic acid activity.

However the information disclosed herein may also be used to reduce chlorogenic acid activity by down-regulation of HQT in cells in which it is desired to do so, thereby having the opposite effect. This may be desirable, for example, for altering texture or palatability of a host plant.

Down-regulation of HQT expression may, for instance, be achieved using anti-sense technology.

In one embodiment, antisense technology may be used to down-regulate HQT expression to inhibit flux to chlorogenic acid so that there will be an increase in ferulic acid which, if cross-linked in the cell walls, will give rise to firmer, crunchier, better-textured plant tissues.

As with plants overexpressing HQT, plants in which HQT expression is downregulated may be produced and analysed under different environmental conditions to investigate the antioxidant function of HQT in plants.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Rothstein et al, 1987; Smith et al,(1988) Nature 334, 724-726; Zhang et al,(1992) The Plant Cell 4, 1575-1588, English et al., (1996) The Plant Cell 8, 179-188. Antisense technology is also reviewed in Bourque, (1995), Plant Science 105, 125-149, and Flavell, (1994) PNAS USA 91, 3490-3496.

Thus, as described above, a nucleotide sequence which is complementary to any of those coding sequences disclosed above forms a further aspect of the present invention.

An alternative to anti-sense is to use a copy of all or part of the target gene inserted in the sense orientation, to achieve reduction in expression of the target gene by co-suppression. See, for example, van der Krol et al., (1990) The Plant Cell 2, 291-299; Napoli et al., (1990) The Plant Cell 2, 279-289; Zhang et al., (1992) The Plant Cell 4, 1575-1588, and U.S. Pat. No. 5,231,020. Further refinements of the co-suppression technology may be found in WO95/34668 (Biosource); Angell & Baulcombe (1997) The EMBO Journal 16,12:3675-3684; and Voinnet & Baulcombe (1997) Nature 389: pg 553.

Another alternative to antisense is ‘gene silencing’ by RNA interference (RNAi), in which double stranded RNA (dsRNA) is used to block gene expression. The dsRNA is cleaved into fragments known as interfering RNAs (iRNAs), which direct the degradation of mRNAs with the same sequence as the iRNAs. Hence, dsRNA comprising the sequence of a target gene can be used to prevent expression of that gene.

For gene silencing of HQT, the inventors have cloned parts of the cDNA of HQT in an iRNA binary vector using the GATEWAY™ system available from Invitrogen (see Example 7) for use in stable and transient transformation of tobacco and tomato plants as described herein.

Further options for down regulation of gene expression include the use of ribozymes, e.g. hammerhead ribozymes, which can catalyse the site-specific cleavage of RNA, such as mRNA (see e.g. Jaeger (1997) ‘The new world of ribozymes’ Curr Opin Struct Biol 7:324-335, or Gibson & Shillitoe (1997)‘Ribozymes: their functions and strategies for their use’ Mol Biotechnol 7: 242-251.)

The complete sequence corresponding to the coding sequence (in reverse orientation for anti-sense) need not be used. For example fragments of sufficient length may be used. It is a routine matter for the person skilled in the art to screen fragments of various sizes and from various parts of the coding sequence to optimise the level of anti-sense inhibition. It may be advantageous to include the initiating methionine ATG codon, and perhaps one or more nucleotides upstream of the initiating codon. A further possibility is to target a conserved sequence of a gene, e.g. a sequence that is characteristic of one or more genes, such as a regulatory sequence.

The sequence employed may be about 500 nucleotides or less, possibly about 400 nucleotides, about 300 nucleotides, about 200 nucleotides, or about 100 nucleotides. It may be possible to use oligonucleotides of much shorter lengths, 14-23 nucleotides, although longer fragments, and generally even longer than about 500 nucleotides are preferable where possible, such as longer than about 600 nucleotides, than about 700 nucleotides, than about 800 nucleotides, than about 1000 nucleotides or more.

It may be preferable that there is complete sequence identity in the sequence used for down-regulation of expression of a target sequence, and the target sequence, although total complementarity or similarity of sequence is not essential. One or more nucleotides may differ in the sequence used from the target gene. Thus, a sequence employed in a down-regulation of gene expression in accordance with the present invention may be a wild-type sequence (e.g. gene) selected from those available, or a variant of such a sequence.

The sequence need not include an open reading frame or specify an RNA that would be translatable. It may be preferred for there to be sufficient homology for the respective anti-sense and sense RNA molecules to hybridise. There may be down regulation of gene expression even where there is about 5%, 10%, 15% or 20% or more mismatch between the sequence used and the target gene. Effectively, the homology should be sufficient for the down-regulation of gene expression to take place.

Thus the present invention further provides the use of the nucleotide sequence of FIG. 7, or its complement, or a variant of either for down-regulation of gene expression, particularly down-regulation of expression of the HQT gene or a homologue thereof, preferably in order to influence the levels of chlorogenic acid in a plant.

Anti-sense or sense regulation may itself be regulated by employing an inducible promoter in an appropriate construct.

The present invention also encompasses the expression product of any of the coding (sense) nucleic acid sequences disclosed above, and methods of making the expression product by expression from encoding nucleic acid therefore under suitable conditions, which may be in suitable host cells.

Following expression, the product may be isolated from the expression system (e.g. microbial) and may be used as desired.

Preferably, the product will have the sequence of one of the polypeptides shown in FIG. 3.

Purified HQT or variant protein, produced recombinantly by expression from encoding nucleic acid therefor, may be used to raise antibodies employing techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and might be screened, preferably using binding of antibody to antigen of interest. For instance, Western blotting techniques or immunoprecipitation may be used (Armitage et al, 1992, Nature 357: 80-82). Antibodies may be polyclonal or monoclonal. As an alternative or supplement to immunising a mammal, antibodies with appropriate binding specificity may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Specific binding members such as antibodies and polypeptides including antigen binding domains of antibodies that bind and are preferably specific for HQT or variant or derivative thereof represent further aspects of the present invention, as do their use and methods which employ them.

All documents cited herein are hereby incorporated by reference inasmuch as is required for the skilled person to carry out the invention.

The invention will now be further described with reference to the following non-limiting Figures and Examples. Other embodiments of the invention will occur to those skilled in the art in the light of these.

### FIGURES

FIG. 1 describes the purification of HQT from Nicotiana tabacum.

FIG. 2 shows how the tomato HQT sequence was assembled from tomato EST clones. Peptide sequences obtained from Q-TOF analysis of the purified HQT protein were identified as parts of the tobacco EST clone AB 0015550 using Blast search analysis. Proceeding from this EST clone, homologous and overlapping tomato EST clones were found and the full-length cDNA of HQT from tomato assembled.

FIG. 3 is an alignment of HQT from tobacco and tomato using MultiAlin (Corpet), 1988). Black background represents highly conserved regions of identical or similar amino acid residues, while they grey background indicates less conserved regions.

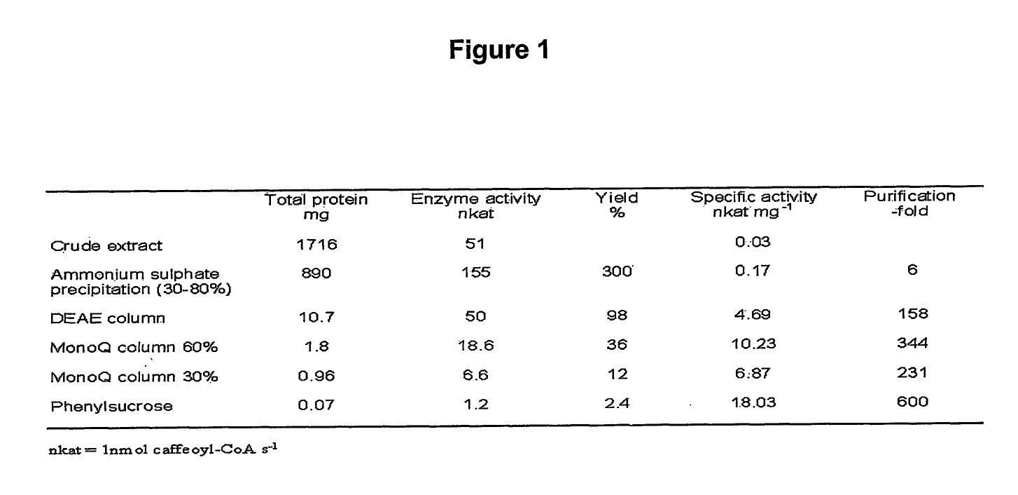

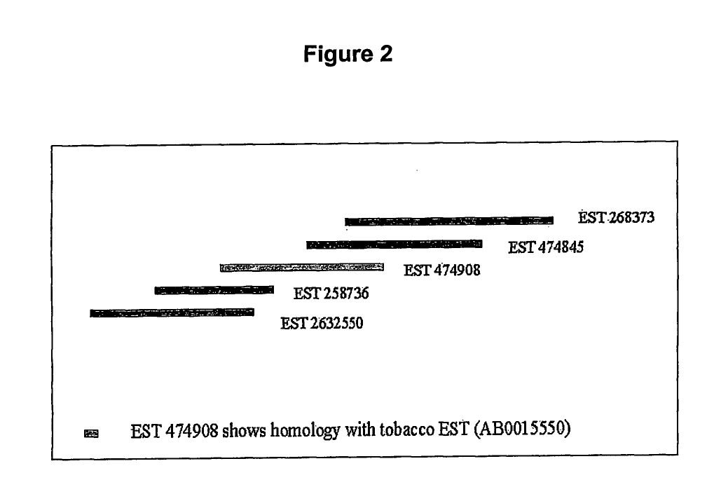

The consensus sequence of the CoA-transferase family (‘cons’) is shown below the HQT sequences. The conserved amino acid motif which is involved in the interaction with the CoA moiety of the substrate is boxed.

FIG. 4 is a phylogenetic tree of HQT proteins from tomato and tobacco. Tomato and tobacco HQT and the related sweet potato protein are shown at the far right. Other branches of the tree represent different proteins of the COA transferase family. The proteins represented are, in clockwise order from Hsrp201:
Hypersensitive response protein 201 from Nicotiana tabacum
Anthranilate N-hydroxycinnamoyl/benzoyl transferase T10711/T10717 from Dianthus caryophyllus
Tomato HQT from Lycopersicon esculentum
Tobacco HQT from Nicotiana tabacum
N-hydroxycinnamoyl/benzoyl transferase from Ipomoea batatas
Anthranilate N-hydroxycinnamoyl/benzoyl transferase from Arabidopsis thaliana
N-hydroxycinnamoyl/benzoyl transferase Arabidopsis thaliana
Putative anthranilate N-hydroxycinnamoyl/benzoyl transferase from Arabidopsis thaliana

FIG. 5 shows the expression of hsr201 and tomHQT in Escherischia coli.
Panel (a) shows Western blot analysis of crude protein extracts from E. coli expressing hsr201 or tomHQT compared to extracts from E. coli transformed with control plasmid (pSTAG). The protein was detected using S-protein alkaline phosphates conjugate.
Panel (b) shows an HQT enzyme assay with crude extracts from E. coli expressing hrs201 or tom HQT. The reaction was initiated by adding the substrate quinic acid and the enzyme activity was measured spectrophotometrically as decrease of the amount of caffeoyl-CoA.

FIG. 6 demonstrates transient expression of HQT from tomato and tobacco in leaves of benthamiana.
Panel (a) shows expression of GFP in N. benthamiana leaves. Leaves were infiltrated with Agrobacterium tumefaciens C58C1 transformed with the plasmid pGFP, which contains the cDNA encoding GFP under control of the 35S promoter, along with bacteria transformed with either the plasmid p19 which carries a cDNA encoding a gene-silencing protein or the control plasmid pBIN19. The expression of GFP was monitored under UV light.
Panel (b) shows the amount of chlorogenic acid detected in leaves infilitrated with Agrobacterium tumefaciens transformed with pGFP, the plasmid pTom which carries the tomato HQT cDNA, or the plasmid pTob which carries the tobacco HQT cDNA, along with bacteria transformed with either p19 or pBIN19 as indicated. The amount of chlorogenic acid in the infiltrated leaf material was measured by HPLC analysis after 4 days.

FIG. 7 shows the cDNA nucleotide sequences of tomHQT (7a) and tobHQT (7b). The transcriptional start and stop codons are indicated in bold and the coding sequence is underlined.

### SEQUENCES

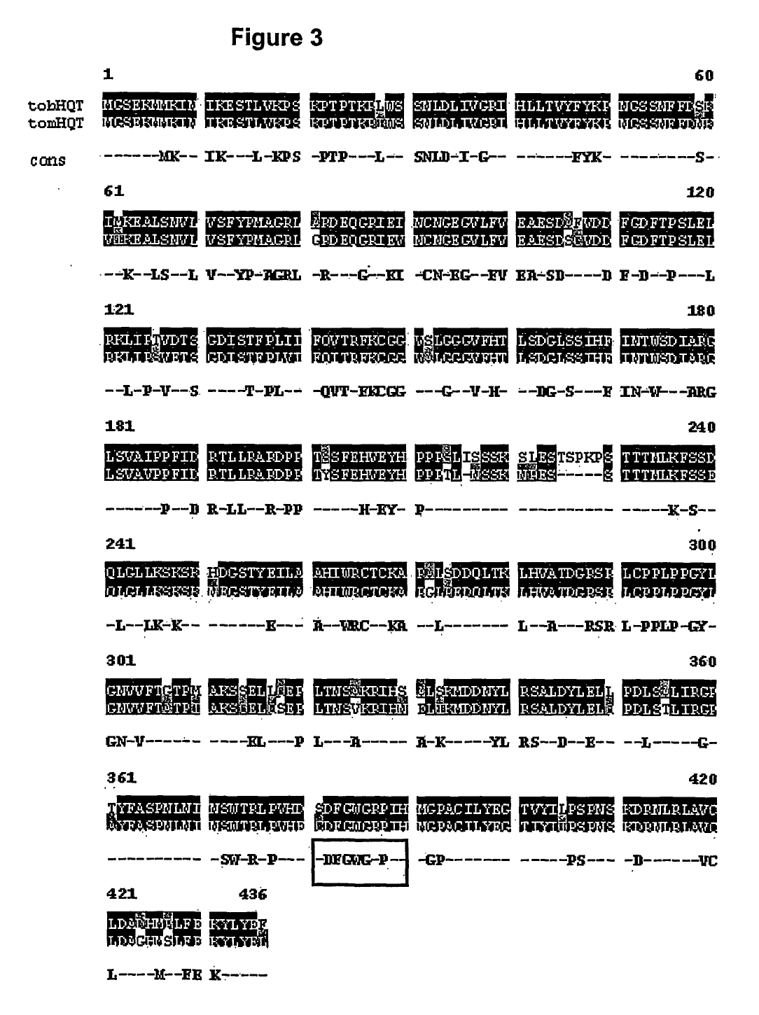

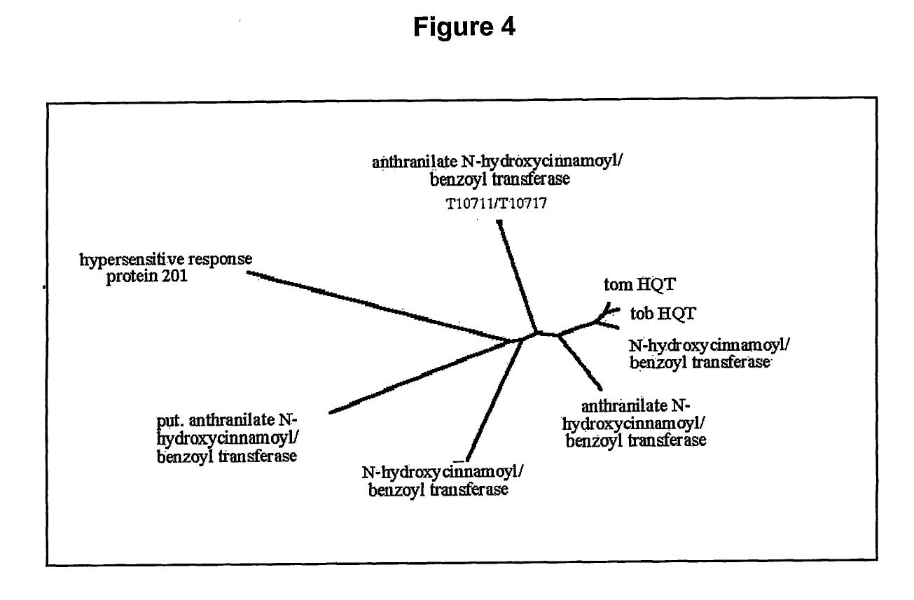

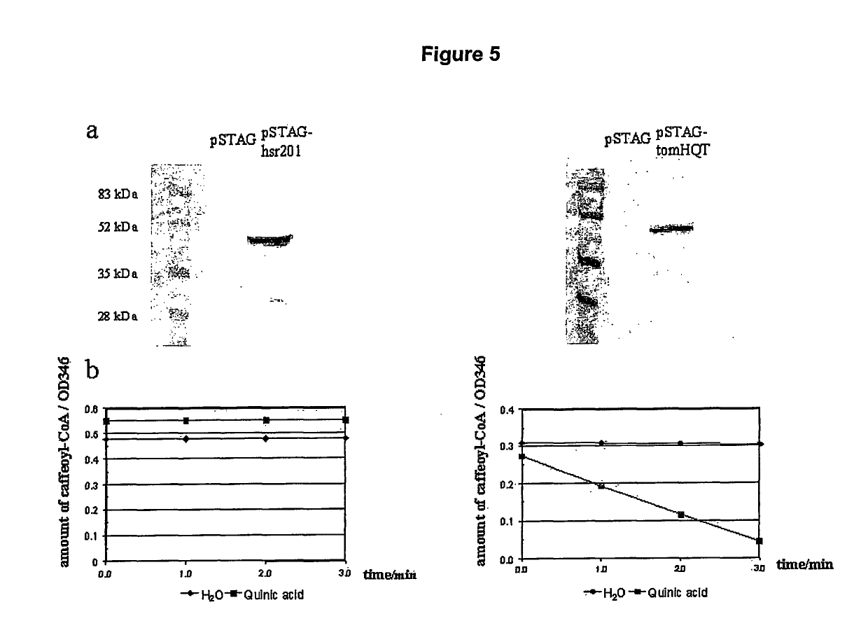

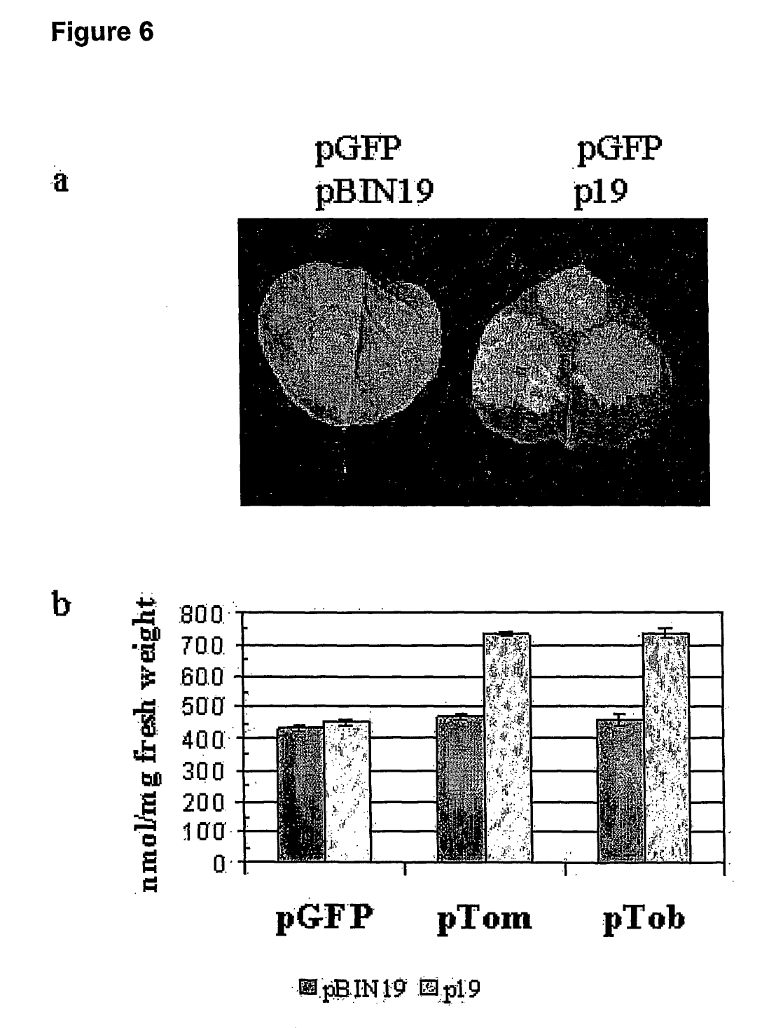

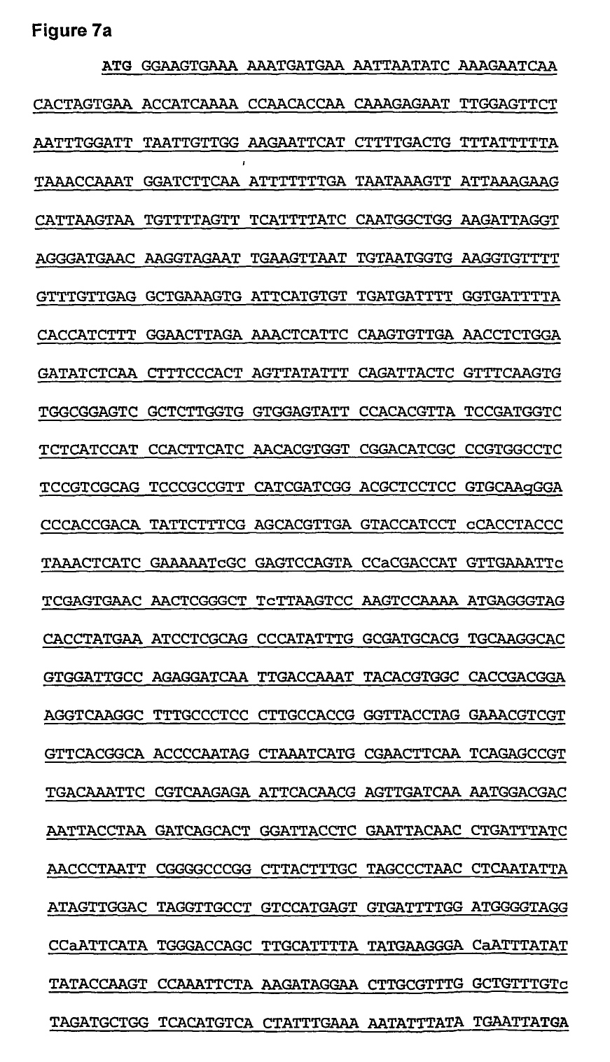

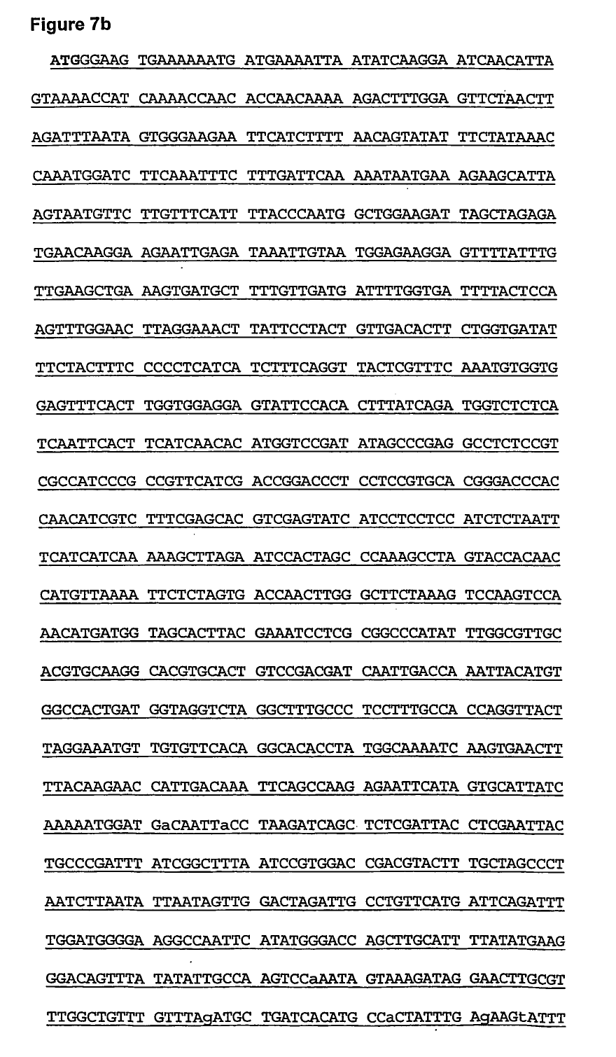

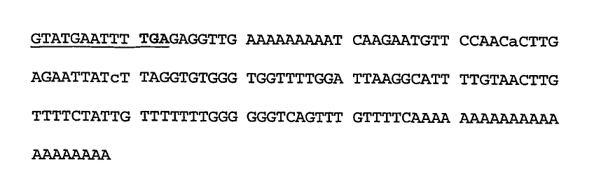

SEQ ID NO 1 is the amino acid sequence of tomato HQT shown in FIG. 3
SEQ ID NO 2 is the amino acid sequence of tobacco HQT shown in FIG. 3
SEQ ID NO 3 is the nucleotide sequence of tobacco HQT shown in FIG. 7b
SEQ ID NO 4 is the nucleotide sequence of tomato HQT shown IN FIG. 7a

### EXAMPLES

### Example 1

Enzyme Extraction and Purification

200 g frozen leaf material from Nicotiana tabacum Samsun NN was ground to a homogenous powder in liquid nitrogen using mortar and pestle. All further purification steps were carried out at 4° C.

The powder was then transferred to 600 ml extraction buffer (0.1 M K-Pi buffer containing 1.8% PVPP, 1% PEG 6000, 1.2% ascorbic acid, 20 μg ml⁻¹ α2-macroglobulin and 5 mM DTT) and stirred until a homogenous solution was obtained. After shaking for 20 min the slurry was passed through a double layer of Miracloth and the filtrate was centrifuged at 25,000 g for 30 min. The supernatant was stirred for 20 min with 10% Dowex 1×2 100-200 which had previously been equilibrated with 0.1 M K-Pi buffer containing 1.2% ascorbic acid, 1% PEG 6000 and 5mM DTT. After centrifugation at 13,000 g for 10 min protamine sulphate (0.1 mg per 1 mg protein) was added to the supernatant. The slurry was then stirred for 20 min and the precipitated protein was collected by centrifugation (30 min at 25,000 g).

The supernatant was made up to 30% satn with finely ground (NH₄)₂SO₄ and stirred for 30 min. The resultant precipitated protein was then removed by centrifugation for 30 min at 25,000 g and the supernatant was raised to 80% satn.

After stirring for 30 min the precipitated protein was pelleted by centrifugation at 25,000 g for 1 h and the pellet was dissolved in 100 ml 10 mM Tris-HCl buffer pH 7.5 containing 5 mM DTT. The protein solution was desalted by dialysis against 10 mM Tris-HCl buffer pH 7.5 overnight.

The protein extract was chromatographed on a DEAE-sepharose (Pharmacia) column (26 cm×2.6 cm), which had been equilibrated with 10 mM Tris-HCl buffer pH 7.5. A gradient of 0-1M KCl in 10 mM Tris-HCl pH 7.5 was applied at a flow rate of 1 ml min⁻¹ and 10 ml fractions were collected. The fraction with the highest enzyme activity was dialysed against 10 mM Tris-HCl pH7.5 for 4 h and then applied to a MonoQ (Pharmacia) column (1 ml), pre-equilibrated in 10 mM Tris-HCL pH 7.5. The proteins were eluted by applying a gradient of 0-600 mM KCL in 10 mM Tris-HCl pH7.5 at a flow rate of 0.5 ml min⁻¹ and 1 ml samples were collected. The two fractions with the highest enzyme activity were pooled and diluted 1:10 with 10 mM Tris-HCl pH 7.5 to reduce the salt concentration. After application onto a MonoQ column the proteins were eluted using a gradient of 0-300 mM KCl in 10 mM Tris-HCl at a flow rate of 0.5 ml min⁻¹ and 1 ml fractions were collected. The fraction with the highest enzyme activity was diluted 1:10 with 10 mM Tris-HCl pH 7.5 containing 1.7 M (NH₄)₂SO₄. The protein extract was then chromatographed on a phenylsucrose (Pharmacia) column (1 ml), pre-equilibrated in 10 mM Tris-HCl pH 7.5/1.7 M (NH₄)₂SO₄, using a 0-100 10 mM Tris-HCl pH 7.5 gradient at a flow rate of 0.25 ml min⁻¹ and 1 ml fractions were collected. The fraction with the highest enzyme activity was concentrated and desalted for polyacrylamide gel electrophoresis (SDS-PAGE) using Microcon YM-10 centrifugal filter devices (Millipore) pH 7.5.

SDS-PAGE was carried out using Tris-glycine pH 8.5 as the buffer system with a 10% acrylamide gel (LAEMMLI, 1970). The 10-fold concentrated enzyme extract was applied and the electrophoresis was carried out at 20 mA. The proteins were visualised using Coomassie Blue. The band containing the putative enzyme was cut out and used for Q-TOF analysis (IFR and JIC Joint Proteomics Facility).

### Example 2

Enzyme Activity

The enzyme activity was determined by spectrophotometry at 30° C. The reaction mixture contained in a total volume of 1 ml: 100 mM Na-Pi buffer pH 7.0, 1 mM EDTA, 16.8 μM caffeoyl CoA and 10-50 μl protein extract. After initiation of the reaction by addition of 2 mM quinic acid the change of caffeoyl CoA into chlorogenic acid could be determined by measuring the rate of decrease in absorbance at 346 nm per minute over a period of 5-10 min. The extinction coefficient for caffeoyl CoA is 1.8×10⁴ M⁻¹ cm⁻¹ (ZENK et al., 1980). The synthesis of caffeoyl CoA was carried out as described previously (SEMLER et al., 1987).

### Example 3

Characterisation of HQT Enzyme Reaction Product Chlorogenic Acid

The reaction mixture was acidified to pH 2.0 with HCl and then extracted twice with 5 ml ethylacetate. After evaporation under vacuum at 40° C. the pellet was dissolved in 200 μl 50% methanol and injected into the HPLC apparatus. The HPLC analysis was carried out as previously described (WALDRON et al., 1996). The reaction product was identified by comparison with the chromatograph of chlorogenic acid (Sigma).

### Example 4

Isolation of Full-Length cDNA of HQT by RT-PCR and 3′ RACE

To isolate the full-length cDNA of HQT from tomato RT-PCR was carried out using the oligonucleotide B26: 5′-GACTCGAGTCGACATCGA(dT)17-3′ for the RT reaction (FROHMAN et al., 1988), and the oligonucleotides EST tom U: 5′-CCATGGGAAGTGAAAAAATGATGAAAA TTAATATC-3′ (corresponding to nucleotides 128 to 160 of the tomato EST clone EST263250 and introducing a NcoI-site in front of the ATG) and EST tom L: 5′-GGATCCTCATAATTCATATAAATATTTTTCAAATA-3′ (corresponding to nucleotides 472 to 500 of the tomato EST clone EST268373 and introducing a BamHI-site after the Stop-Codon) for the PCR reaction. The EST clones were identified by BLAST search using the peptide sequences obtained from Q-TOF analysis of the purified enzyme.

5 μg total RNA isolated from tomato leaves was used for the RT reaction which was carried out using the SUPERSCRIPT™ First-Strand Synthesis System according to the procedures provided by the manufacturer (GIBCO BRL).

The PCR reaction was conducted at 94° C. for 40 sec, 55° C. for 2 min, 72° C. for 3 min in 40 cycles, and 72° C. for 10 min. The reaction mixture contained 10 μl RT-reaction mixture (1:50 diluted), 1×reaction buffer without MgSO₄, 0.2 mM dNTPs, 0.25 μM EST tom U, 0.25 μM EST tom L, 5% DMSO, 3 mM MgSO₄ and 2.5 U Pwo DNA-Polymerase (Roche) in a final volume of 100 μl.

The isolation of the cDNA of HQT from tobacco was conducted in two steps:
by 3′-RACE to amplify the C-terminal part of the cDNA using the oligonucleotide B26: 5′-GACTCGAGTCGACATCGA(dT)17-3′ for the RT reaction, and the oligonucleotides EST tob inter: 5′-GAGCACGTCGAGTATCATCCTCCTC CATC-3′ (corresponding to nucleotides 6 to 34 of the tobacco EST clone AB001550), the nested oligonucleotid EST tob inter2 U: 5′-CTAATTTCATCATCAAAAAGCTTAGAATCCAC-3′ (corresponding to nucleotides 36 to 66 of the tobacco EST clone AB001550 and introducing a Hind III-site) and B25: 5′-GACTCGAGTCGACATCG-3′ for the PCR reaction;
by RT-PCR to amplify the N-terminal part of the cDNA using the oligonucleotide B26: 5′-GACTCGAGTCGACATCGA(dT)17-3′ for the RT reaction, and the oligonucleotides EST tom U and EST tob inter2 L: 5′-GTGGATTCTAAGCTTTTTGATGATGAAATTAG-3′ (corresponding to nucleotides 36 to 66 of the tobacco EST clone AB001550 and introducing a Hind III-site) for the PCR reaction. 5 μg total RNA isolated from tobacco leaves were used for the RT reaction which was carried out using the SUPERSCRIPT™ First-Strand Synthesis System according to the manufacturers (GIBCO BRL) instructions.

The 3′-RACE-PCR reaction was conducted at 94° C. for 40 sec, 55° C. for 2 min, 72° C. for 3 min in 40 cycles, and 72° C. for 10 min. The reaction mixture contained 10 μl RT-reaction mixture (1:50 diluted), 1×reaction buffer without MgSO₄, 0.2 mM dNTPs, 0.25 μM EST tob inter, 0.25 μM B25, 2 mM MgSO₄ and 2.5 U Pwo DNA-Polymerase (Roche) in a final volume of 100 μl. The PCR products were diluted 500-fold and they were then used in a second PCR reaction, which was carried out using the nested oligonucleotide EST tob inter2 and B25 under the some conditions.

To amplify the N-terminal part of the cDNA, a PCR reaction was conducted at 94° C. for 40 sec, 45° C. for 2 min, 72° C. for 2 min in 10 cycles, followed by 30 cycles at 94° C. for 40 sec, 55° C. for 2 min, 72° C. for 2 min, and finally at 72° C. for 10 min. The reaction mixture contained 10 μl RT-reaction mixture (1:50 diluted), 1×reaction buffer without MgSO₄, 0.2 mM dNTPs, 0.25 μM EST tob inter2 L, 0.25 μM EST tom U, 3 mM MgSO₄, 5% DMSO and 2.5 U Pwo DNA-Polymerase (Roche) in a final volume of 100 μl.

After cloning of the PCR products into the plasmid pGEM®T easy, according to the procedures provided by the manufacturer (Promega), the products were sequenced using the ABI Prism™ Dye Terminator Cycle Sequencing Ready Reaction Kit according to the procedures provided by the manufacturer (Perkin Elmer).

The full-length cDNA of HQT from tobacco was obtained by assembling the two PCR products using the HindIII site that had been introduced within the sequence of the oligonucleotides used in the PCR reaction.

### Example 5

Recombinant Expression of HQT in E. coli

The full-length cDNA of HQT from tomato was subcloned in frame into the NcoI and BamHI-sites of the expression vector pSTAG under the control of the T7 RNA-Polymerase promoter. This strategy led to the introduction of a sequence encoding an S-TAG at the 5′-end of the cDNA. According to procedures provided by the manufacturer (Novagen), the expression and purification of the fusion protein was carried out using the S-TAG™ Thrombin Purification Kit. The enzyme activity of the purified protein was then analysed in HQT enzyme assays.

### Example 6

HPLC of Soluble Phenolics in Nicotiana benthamiana Leaves

Soluble phenolics were extracted by vortexing 30 mg of frozen, powdered leaf material in 300 μl of 70% methanol. After addition of 100 μl distilled water, the extract was vortexed again and then spun for 10 sec at 13,000 g to pellet the debris. The supernatant was analysed by gradient HPLC (apparatus from Spectra Physics, San Jose, Calif.) using a Columbus 5-μm C18 column (25 cm×4.6 mm; Phenomenex, Macclesfiled, Cheshire, UK). Solvent A was 1 mM trifluoroacetic acid, solvent B was acetonitrile, and the gradient was run for 65 min at flow rate of 1 ml min⁻¹ with the following concentrations: 0 min :100% A, 0% B; 50 min: 60% A, 40% B; 60 min: 30% A, 70% B; 65 min: 100% A, 0% B. The column eluant was monitored using a Spctra Fous scanning detector (Spectra Physics). A chlorogenic acid standard (Sigma) was used to quantify the amount of chlorogenic acid in the leaf extracts.

### Example 7

Constructs for Overexpression and Gene Silencing of HQT in Plants

For overexpression of HQT, the full-length cDNA of HQT from tomato and tobacco was subcloned in frame into the NcoI and SmaI-sites of the expression vector pJIT166 (available from JIC) under the control of the double 35S-promoter. The expression cassette containing the promoter, the cDNA and the CaMV terminator was then cloned in the binary vector pBIN19 (BEVAN, 1984). The obtained constructs pTom and pTob were used for transient and stable transformation of tomato and tobacco plants in order to achieve overexpression of HQT

For gene silencing of HQT, to obtain iRNA constructs 600 bp cDNA-fragments of HQT from tobacco and tomato were amplified by PCR using the oligonucleotides G-tom sil U: 5′-GGGGACAAGTTTGTACAAAAAAGCAGGCTCAGATTACTCGTTTCAAGTGTGGC-3,′, G-tom sil L: 5′-GGGGACCACTTTGTACAAGAAAGCTGGGTTGATAA ATCAGGTTGT AATTCGAGG-3′ (corresponding to the nucleotides 424 to 447 and 1020 to 1044 of the tomato cDNA clone) and G-tob sil U: 5′-GGGGACAAGTTTGTACAAAAAAGCAGGCTCAAATGTGGTGGAGTTTCACTG-3′, G-tob sil L: 5′-GGGGACCACTTTGTACAAGAAAGCTGGGTCGGGCAGT AATTCGAGGTAATC (corresponding to the nucleotides 438 to 460 and 1034 to 1056 of the tobacco cDNA clone). The cloning of the PCR fragments into the vector pDONR201 was carried out using the GATEWAY™ Cloning system, according to the procedures provided by the manufacturers (INVITROGEN). After sequencing the fragments were cloned in both directions into the RNAi plasmid pFRN (kindly provided by M. Denekamp), which contains attR recombination site in sense and antisense orientation spaced by an intron to enable the formation of double stranded RNA. The production of dsRNA is driven by the 35S promoter. The constructs pFRN-tom and pFRN-tob were used for stable and transient transformation of tomato and tobacco plants.

### Example 8

Stable Transformation of Tobacco and Tomato Plants

The pBIN19-derivatives pTom and pTob, which contain the full-length cDNA of HQT from tomato and tobacco under the control of the double 35S, and the iRNA constructs pFRN-tom and pFRN-tob were transferred to Agrobacterium tumefaciens LBA 4404 (HOEKEMA et al., 1983). A single, transformed colony was used to obtain a 50 ml overnight culture. After being pelleted by centrifugation for 20 min at 4000 g the cells were resuspended in 50 ml MS medium and used for transformation of leaf discs from Nicotiana tabacum var Samsun NN (HORSCH et al., 1985) and of cotyledons from Lycopersicon esculentum var Moneymaker (FILLATI et al., 1987). Developing shoots were cut and transferred onto MS medium containing 100 ug/ml Kanamycin and 500 ug/ml Cefotaxim (tobacco) or 200 ug/ml Augmentin (tomato) for rooting.

### Example 9

Transient Expression of HQT in Leaves of Tobacco Plants

After the transfer of the overexpression constructs pTom and pTob, and the iRNA constructs pFRNtom and pFRN-tob to Agrobacterium tumefaciens C58C1 (DEBLAERE et al., 1985) single colonies were used for growing 50 mL overnight cultures. Additional 50 ml overnight cultures of Agrobacterium tumefaciens C58C1 containing expression constructs of the green fluorescent protein (PGFP) or the gene silencing inhibitor protein 19 (p19; kindly provided by O. Vionnet) or the empty binary vectors pBIN19 and pFRN were also grown. The cells were precipitated by centrifugation at 4000 g for 20 min and resuspended in 50 ml 10 mM MgCl₂. After addition of 50 μl 100 mM acetosyringene the cells were incubated at room temperature overnight. To overexpress HQT similar amounts of the cultures containing pGFP, pTom, pTob were mixed with the same volume of cultures containing either pBIN19 or p19 and each mixture was used to infiltrate three leaves of a Nicotiana benthamiana plant. After 4 days, when the expression of GFP could be monitored under UV light, the leaf material was collected and used for quantification of the chlorogenic acid content by HPLC analysis. For gene silencing the cultures containing pFRN-tom, pFRN-tob and pFRN were used to infiltrate three leaves of Nicotiana benthamiana. After 7 days the leaf material was collected and also analysed by HPLC to measure the chlorogenic acid content.

### REFERENCES

BEVAN, M. (1984): Binary Agrobacterium vectors for plant transformation. Nucl. Acids Res. 12, 8711-8721
BRAVO, L. (1998): Polyphenols: Chemistry, dietary sources, metabolism, and nutritional significance. Nutrition Reviews 56, 317-333
CASTELLUCCIO, C., PAGANGA, G., MELIKIAN, N., BOLWELL, G. P., PRIDHAM, J., SAMPSON J. and RICE-EVANS, C. (1995): Antioxidant potential of intermediates in phenyl propanoid metabolism in higher plants. FEBS Lett. 368, 188-192
CORPET, F. (1988): Multiple sequence alignment with hierarchical clustering. Nucleic Acids Research 16 10881-10890
DEBLAERE, R., BYTEBIER, B., DE-GREVE, H., DEBOECK, F., SCHELL, J., VAN-MONTAGU, M. and LEEMANS, J. (1985): Efficient octopine Ti plasmid-derived vectors for Agrobacterium-mediated gene transfer to plants. Nucl. Acids Res. 13, 4777-4778
DEL MORAL, R. (1972): On the variability of chlorogenic acid concentration. Oecologia 9, 289-300
DIAZ, J., BARCELO, A. R. and DE CACERES, F. M. (1997): Changes in shikimate dehydrogenase and the end products of the shikimate pathway, chlorogenic acid, and lignins, during the early development of seedlings of Capsicum annum. New Phytol. 136, 183-188
DIXON, R. A. and PAIVA, N. L. (1995): Stress-induced phenylpropanoid metabolism. Plant Cell 7, 1085-1097
FILLATI, J. J., KISER, I., ROSE, S. and COMAI, L. (1987): Efficient transfer of a glyphosphate tolerance gene into tomato using a binary Agrobacterium tumefaciens vector. Bio/Technology 5, 726-730
FROHMAN, M. A., DUSH, M. K. and MARTIN, G. R. (1988): Rapid production of full-length cDNAs from rare transcripts: Amplification using single gene-specific oligonucleotide primer. Proc. Natl. Acad. Sci. USA 85, 8998-9002
GRACE, S. C. and LOGAN, B. A. (2000): Energy dissipation and radical scavenging by the plant phenylpropanoid pathway. Phil. Trans. R. Soc. Lond. B 355, 1499-1510
GRACE, S. C., LOGAN, B. A. and ADAMS, W. W. (1998): Seasonal differences in foliar content of chlorogenic acid, a phenylpropanoid antioxidant, in Mahonia repens. Plant Cell Environ. 21, 513-521
HOEKEMA, A., HIRSCH, P. R., HOOYKAS, P. J. J. and SCHILPERPOORT, R. A. (1983): A binary plant vector strategy based on separation of the vir and T-region of the Agrobacterium tumefaciens Ti-plasmid. Nature 303, 179-180
HORSCH, R. B., FRY, J. E., HOFFMANN, D., EICHHOLTZ, D., ROGERS, S. G. and FRALEY, R. T. (1985): A simple and general method for transferring genes into plants. Science 227, 1229-1231
KONO, Y., KOBAYASHI, K., TAGAWA, S., ADACHI, K., UEDA, A., SAWA, Y. and SHIBATA, H. (1997): Antioxidant activity of polyphenolics in diets. Rate constants of reactions of chlorogenic acid and caffeic acid with reactive species of oxygen and nitrogen. Biochim. Biophys. Acta 1335, 335-342
KUEHNL, T., KOCH, K., HELLER, H. and WELLMANN, E. (1987): Chlorogenic acid biosynthesis: Characterization of a light-induced microsomal 5-O-(4-coumaroyl)-D-quinate/shikimate 3′-hydroxylase from carrot (Daucus carota L.) cell suspension cultures. Arch. Biochem. Biophys. 258, 226-232
LAEMMMLI, U. K. (1970): cleavage of structural proteins during the assembly of the head of bacteriophage T4. Nature 227, 680-685
LOTFY, S., FLEURIET, A. and MACHEIX, J. J. (1992): Partial purification and characterization of hydroxycinnamoyl CoA:transferases from apple and date fruits. Phytochem. 31, 767-772
LOTFY, S. (1995): Inactivation and kinetic characterization of hydroxycinnamoyl-CoA:hydroaromatic acid O-hydroxycinnamoyltransferases from Cichorium endivia and Phoenix dactylifera. Plant Physiol. Biochem. 33, 423-431
MACHEIX, J. J., SAPIS, J. C., and FLEURIET, A. (1991): Phenolic compounds and polyphenoloxydase in relation to browning in grapes and wines. Crit. Rev. Food Sci. Nutr. 30, 441-486
RICE-EVANS, C. A., MILLER, N. J. and PAGANGA, G. (1997): Antioxidant properties of phenolic compounds. Trends Plant Science 2, 152-159 RHODES, M. J. C. and WOOLTORTON, L. S. C. (1976) : The enzymic conversion of hydroxycinnamic acids to p-coumaroylquinic and chlorogenic acids in tomato fruits. Phytochem 15, 947-951
RHODES, J. M. and WOOLTORTON, L. S. C. (1978): The biosynthesis of phenolic compounds in wounded plant storage tissues. In Biochemistry of Wounded Plant Tissues. Walter de Gruyter & Co., Berlin, 243-286
RHODES, M. J., WOOLTORTON, L. S. and LOURENCO, E. J. (1979): Purification and properties of hydroxycinnamoyl CoA quinate hydroxycinnamoyl transferase from potatoes. Phytochem. 18, 1125-1129
SCHOCH, G., GOEPFERT, S., MORANT, M., HEHN, A., MEYER, D., ULLMANN, P. and WERCK-REICHHART (2001): CYP98A3 from Arabidopsis thaliana is a 3′-hydroxylase of phenolic esters, a missing link in the phenylpropanoid pathway. J Biol. Chem. 276, 36566-36574
SEMLER, U., SCHMIDTBERG, G. and GROSS, G. G. (1987): Synthesis of piperoyl coenzyme-A thioester. Z NATURFORSCH 42, 1070-1074
STOECKIGT, J. and ZENK, M. H. (1974): Enzymatic synthesis of chlorogenic acid from caffeoyl coenzyme A and quinic acid. FEBS Lett. 42, 131-134
STRACK, D. (1997): Phenolic metabolism. In Plant Biochemistry, p387-417. Academic Press Ltd
ULBRICH, B. and ZENK, M. H. (1979): Partial purification and properties of hydroxycinnamoyl-CoA:quinate hydroxycinnamoyl transferase from higher plants. Phytochem. 19, 1625-1629
VILLEGAS, R. J. A. and KOJIMA, M. (1986): Purification and characterization of hydroxycinnamoyl D-glucose quinate hydroxycinnamoyl transferase in root of sweet potato. J Biol. Chem. 261, 8729-8733
WALDRON, K. W., PARR A. J., NG A. and RALPH J. (1996): Cell wall esterified phenolic dimers: Identification and quantification by reverse phase high performance liquid chromatography and diode array detection. PHYTOCHEMICAL ANALYSIS 7, 305-312
YAO, K., DE LUCA, V. and BRISSON, N. (1995): Creation of a metabolic sink for tryptophan alters the phenylpropanoid pathway and the susceptibility of potato to Phytopthora infestans. Plant Cell 7, 1787-1799
ZENK, M. H., ULBRICH, B., BUSSE, J. and STOECKIGT, J. (1980): Procedure for the enzymatic synthesis and isolation of cinnamoyl-CoA thiolesters using a bacterial system. Analytical Biochemistry 101, 182-187

1. An isolated nucleic acid which comprises a hydroxycinnamoyl-CoA quinate hydroxycinnamoyl transferase (HQT) nucleotide sequence encoding a polypeptide which has HQT activity.

2. A nucleic acid according to claim 1 wherein the HQT nucleotide sequence is obtainable from tomato.

3. A nucleic acid according to claim 1 wherein the HQT nucleotide sequence is obtainable from tobacco.

4. A nucleic acid according to claim 2 wherein the HQT nucleotide sequence encodes a polypeptide with the amino acid sequence shown in SEQ ID No 1.

5. A nucleic acid according to claim 3 wherein the HQT nucleotide sequence encodes a polypeptide with the amino acid sequence shown in SEQ ID No 2.

6. A nucleic acid according to claim 5 wherein the HQT nucleotide sequence has the nucleotide sequence of SEQ ID No 3.

7. A nucleic acid according to claim 6 wherein the HQT nucleotide sequence has the nucleotide sequence of SEQ ID No 4.

8. A nucleic acid comprising a hydroxycinnamoyl-CoA quinate hydroxycinnamoyl transferase (HQT) nucleotide sequence as claimed in claim 1 which is a homologous variant of SEQ ID No 3 or SEQ ID No 4 and which shares at least about 85%, 90%, 95%, 96%, 97%, 98% or 99% identity with either.

9. A nucleic acid according to claim 8 wherein said HQT nucleotide sequence encodes a polypeptide which has HQT activity.

10. A nucleic acid according to claim 8 wherein the HQT nucleotide sequence is a derivative of SEQ ID No 3 or SEQ ID No 4 which differs therefrom by way of one or more of nucleotide addition, deletion or substitution, said nucleic acid optionally encoding a polypeptide having HQT activity.

11. A nucleic acid which comprises a complement HQT nucleotide sequence sequence which is the complement of the HQT nucleotide sequence of claim 1.

12. An isolated nucleic acid for use as primer, said nucleic acid consisting of a sequence as claimed in claim 1, said primer being about 16-32 nucleotides in length, which sequence is present in either the HQT nucleotide sequence encoding the polypeptide of SEQ ID NO: 1 or SEQ ID NO: 2 or the complement thereof.

13. An isolated nucleic acid for use as a probe, said nucleic acid consisting of a sequence as claimed in claim 1, said probe being of at least about 100 nucleotides or more, about 200 nucleotides or more, about 300 nucleotides or more, or about 400 nucleotides or more, which contiguous sequence is present in either the HQT nucleotide sequence of SEQ ID NO: 3 or SEQ ID NO: 4, or the complement thereof.

14. A method for identifying, cloning, or determining the presence of a HQT encoding nucleic acid as claimed in claim 1, which method employs a nucleic acid selected from the group consisting of SEQ ID NO: 3, SEQ ID NO: 4, a homologous variant of SEQ ID No 3 or SEQ ID No 4 and which shares at least about 85%, 90%, 95%, 96%, 97%, 98% or 99% identity with either, a nucleic acid encoding a polypeptide of SEQ ID NO: 1, a nucleic acid encoding a polypeptide of SEQ ID NO: 2, at least one primer of about 16-32 nucleotides in length, which sequence is present in the HQT nucleotide sequence encoding the polypeptide of SEQ ID NO: 1 or SEQ ID NO: 2 or the complement thereof, and a probe of at least about 100 nucleotides or more, about 200 nucleotides or more, about 300 nucleotides or more, or about 400 nucleotides or more, which contiguous sequence is present in either the HQT nucleotide sequence of SEQ ID NO: 3 or SEQ ID NO: 4, or the complement thereof.

15. A method as claimed in claim 14 which comprises the steps of:
(a) providing a preparation of nucleic acid from a plant cell;
(b) providing said nucleic acid molecule;
(c) contacting nucleic acid in said preparation with said nucleic acid molecule under conditions for hybridisation, and,
(d) identifying said HQT encoding nucleic acid if present by its hybridisation with said nucleic acid molecule.

16. A method as claimed in claim 14, which method comprises the steps of:
(a) providing a preparation of nucleic acid from a plant cell;
(b) providing a pair of nucleic acid molecule primers suitable for PCR
(c) contacting nucleic acid in said preparation with said primers under conditions for performance of PCR,
(d) performing PCR and determining the presence or absence of an amplified PCR product.

17. A recombinant vector which comprises an HQT nucleic acid said nucleic acid being selected from the group consisting of SEQ ID NO: 3, SEQ ID NO: 4, a nucleic acid encoding a polypeptide of SEQ ID NO: 1, a nucleic acid encoding a polypeptide of SEQ ID NO: 2, a homologous variant of SEQ ID No 3 or SEQ ID No 4 and which shares at least about 85%, 90%, 95%, 96%, 97%, 98% or 99% identity with either, at least one primer of about 16-32 nucleotides in length, which sequence is present in the HQT nucleotide sequence encoding the polypeptide of SEQ ID NO: 1 or SEQ ID NO: 2 or the complement thereof, and a probe of at least about 100 nucleotides or more, about 200 nucleotides or more, about 300 nucleotides or more, or about 400 nucleotides or more, which contiguous sequence is present in either the HQT nucleotide sequence of SEQ ID NO: 3 or SEQ ID NO: 4, or the complement thereof.

18. A vector as claimed in claim 17 wherein the nucleic acid is operably linked to a promoter for transcription in a host cell, wherein the promoter is optionally an inducible promoter.

19. A vector as claimed in claim 17 which is a plant vector, said vector optionally comprising a promoter sequence.

20. A method which comprises the step of introducing the vector of claim 17 into a host cell, and optionally causing or allowing recombination between the vector and the host cell genome such as to transform the host cell.

21. A host cell containing or transformed with a heterologous nucleic acid of claim 1 such as to alter one or more of the cell's characteristics with respect to chlorogenic acid synthesis.

22. A host cell as claimed in claim 21 which is a plant cell, optionally present in a plant.

23. A method for producing a transgenic plant, which method comprises the steps of:
(a) performing a method as claimed in claim 20,
(b) regenerating a plant from the transformed plant cell.

24. A transgenic plant which is obtainable by the method of claim 23, said transgenic plant comprising a plant cell comprising an HQT nucleic acid.

25. A part of propagule from a plant as claimed in claim 24.

26. An isolated polypeptide which is encoded by the HQT nucleotide sequence of claim 1, said nucleic acid being selected from the group consisting of SEQ ID NO: 3 or SEQ ID NO: 4,a homologous variant of SEQ ID No 3 or SEQ ID No 4 and which shares at least about 85%, 90%, 95%, 96%, 97%, 98% or 99% identity with either, a nucleic acid encoding SEQ ID NO: 1, and a nucleic acid encoding SEQ ID NO: 2.

27. A polypeptide as claimed in claim 26 comprising the amino acid sequence of SEQ ID No 1.

28. A polypeptide as claimed in claim 26 comprising the amino acid sequence of SEQ ID No 2.

29. A polypeptide which comprises the antigen-binding site of an antibody having specific binding affinity for a polypeptide encoded by the nucleic acid of claim 26.

30. A method of making a polypeptide selected from the group consisting of SEQ ID NO: 1, SEQ ID NO: 2, a polypeptide encoded by SEQ ID NO: 3, a polypeptide encoded by SEQ ID NO: 4, a homologous variant of SEQ ID No 3 or SEQ ID No 4 and which shares at least about 85%, 90%, 95%, 96%, 97%, 98% or 99% identity with either which method comprises the step of causing or allowing expression from a nucleic acid of claim 17 in a suitable host cell.

31. A method for influencing chlorogenic acid levels in a plant, which method comprises the step of causing or allowing expression of a heterologous nucleic acid within the cells of the plant, which nucleic acid comprises a HQT nucleotide sequence or the complement thereof, following an earlier step of introducing the nucleic acid into a cell of the plant or an ancestor thereof.

32. A method according to claim 31, wherein the heterologous nucleic acid is a nucleic acid as claimed in claim 1.

33. A method according to claim 31, wherein the heterologous nucleic acid comprises the nucleotide sequence of NCBI accession number AB035183.

34. A method as claimed in claims 31 for increasing the level of chlorogenic acid in a plant to improve any one or more of: flavour; palatability; nutritional value; resistance to abiotic stress; pathogen resistance; antioxidant properties, of the plant, said heterlogous nucleic acid being selected from the group consisting of SEQ ID NO: 3, SEQ ID NO: 4, the nucleotide sequence of NCBI accession number AB035183, a nucleic acid encoding a polypeptide of SEQ ID NO: 1, a nucleic acid encoding a polypeptide of SEQ ID NO: 2, a homologous variant of SEQ ID No 3 or SEQ ID No 4 and which shares at least about 85%, 90%, 95%, 96%, 97%, 98% or 99% identity with either.

35. A method as claimed in claim 32 for reducing the level of chlorogenic acid in a plant to improve any one or more of: flavour; palatability; texture; nutritional value, of the plant, said method being selected from the group consisting of at least one of:
(i) causing or allowing transcription from a nucleic acid which is a derivative of SEQ ID NO: 3 or SEQ ID NO: 4 having one or more nucleic acid additions, deletions or substitutions, in the plant such as to reduce HQT expression by an antisense mechanism, or
(ii) causing or allowing transcription from a nucleic acid selected from the group consisting of SEQ ID NO: 3, SEQ ID NO: 4, the nucleotide sequence of NCBI accession number AB035183, a nucleic acid encoding a polypeptide of SEQ ID NO: 1, a nucleic acid encoding a polypeptide of SEQ ID NO: 2, a homologous variant of SEQ ID No 3 or SEQ ID No 4 and which shares at least about 85%, 90%, 95%, 96%, 97%, 98% or 99% identity with either. or a part thereof such as to reduce HQT expression by co-suppression,
(iii) introducing into said plant a double-stranded RNA corresponding to a nucleotide sequence of step ii),
(iv) introducing into said plant a nucleic acid encoding a ribozyme specific for a nucleic acid of step ii).
